(19) United States
(12) Patent Application Publication
(10) Pub. No.: US 2022/0257646 A1
(43) Pub. Date: Aug. 18, 2022

(54) COMPOSITIONS AND METHODS FOR TREATING SKIN CONDITIONS

(71) Applicant: The Regents of the University of California, Oakland, CA (US)

(72) Inventors: Huiying Li, Los Angeles, CA (US); Emma Barnard, Los Angeles, CA (US)

(21) Appl. No.: 17/628,079

(22) PCT Filed: Jul. 17, 2020
(86) PCT No.: PCT/US2020/042542

§ 371 Date: Jan. 18, 2022

Related U.S. Application Data
(60) Provisional application No. 62/875,641, filed on Jul. 18, 2019.

(51) Int. Cl. A61K 33/26; A61P 17/10; A61K 35/74; A61Q 19/08; A61K 9/00

(52) U.S. Cl. CPC A61K 33/26; A61P 17/10; A61K 9/0014; A61Q 19/08; A61K 9/0053; A61K 35/74

(57) ABSTRACT

Provided herein are methods and compositions for the treatment or prevention of a skin disease (e.g., acne) in a subject by administering to the subject a composition comprising iron or cobalt.

### RELATED APPLICATIONS

This application claims priority to U.S. Provisional Application No. 62/875,641, filed Jul. 18, 2019, which is incorporated herein by reference in its entirety.

### GOVERNMENT SUPPORT

This invention was made with government support under Grant Number GM099530, awarded by the National Institutes of Health. The government has certain rights in the invention.

### BACKGROUND

The skin is the largest organ in the human body and functions as the first line of defense by providing a protective barrier between the environment and inner body. The skin harbors several hundreds of resident microorganisms, which function in communities and protect the body from invasion of pathogens. Several studies have shown that shifts in the skin microbiota are associated with various skin diseases.

Acne vulgaris (commonly called acne) is the most common skin disease, affecting 80-85% of the population. It is most prevalent in adolescents and rarely occurs in people over the age of 50. Although acne is not life threatening, it can lead to severe pain and scarring on the skin, and has profoundly negative psychosocial effects. Acne is a disease of the pilosebaceous unit (commonly known as the hair follicle). While its etiology is still unclear with multiple factors involved, the Gram-positive lipophilic anaerobe Propionibacterium acnes (recently renamed to Cutibacterium acnes) has been thought to play a role in acne pathogenesis. Propionibacterium acnes is a dominant bacterium on human skin.

### SUMMARY

In some aspects, provided herein are method and compositions (e.g., pharmaceutical or cosmetic compositions) useful in treating or preventing a skin disease or skin aging in a subject in need thereof by administering a composition comprising iron and/or cobalt to the subject. In some embodiments, the subject has a skin disease (e.g., inflammatory skin disease, such as acne, rosacea, or Porphyria Cutanea Tarda (PCT)).

In some aspects, provided herein are methods and compositions related to treating or preventing a skin disease (e.g., inflammatory skin disease, such as acne, rosacea, or Porphyria Cutanea Tarda (PCT)), preventing and/or slowing skin aging (e.g., preventing the formation of wrinkles), and maintaining healthy skin by administering to the subject a composition disclosed herein. The method of any one of the preceding claims, wherein the skin disease is an inflammatory skin disease. The skin disease may be acne, rosacea, or PCT.

In some aspects, provided herein are methods of treating or preventing a skin condition in a subject and/or reducing the amount of porphyrins (e.g., bacterially derived porphyrins) on the skin of a subject by administering (e.g., a subject with symptoms of skin aging, or a subject with a skin condition, such as a skin disease associated with inflammation, acne, rosacea, or PCT) a composition disclosed herein to the subject. The porphyrins may be produced by P. acnes (e.g., acne-associated strains of P. acnes). The porphyrins may be produced by P. granulosum, P. avidum, or P. humerusii.

In some aspects, provided herein are methods of promoting vitamin B12 biosynthesis in acne-associated strains of P. acnes on the skin of a subject by administering a compositions comprising cobalt and/or iron. In some embodiments, the subject has symptoms of skin aging. In some embodiments, the subject has a skin condition, such as a skin disease associated with inflammation, acne, rosacea, or PCT.

The compositions disclosed herein may further comprises one or more strains of P. acnes. The P. acnes strain may be RT1, RT2, RT3, or RT6 strain of P. acnes. The compositions may further comprise at least one phage against a strain of P. acnes (e.g., a phage that target a strain of P. acnes (e.g., RT4, RT5, RT7, RT8, RT9 or RT10). The compositions may further comprise P. granulosum, P. avidum, P. humerusii. Compositions disclosed herein may comprise an antibiotic (e.g., an antibiotic that does not target P. granulosum, P. avidum, P. humerusii, or an RT1, RT2, RT3, or RT6 strain of P. acnes).

In certain embodiments, the compositions disclosed herein may be formulated for oral or topical delivery. The subject may be a human subject.

### BRIEF DESCRIPTION OF THE FIGURES

FIG. 1 shows that P. acnes strains respond to iron supplemented in the media and addition of iron leads to low porphyrin production.

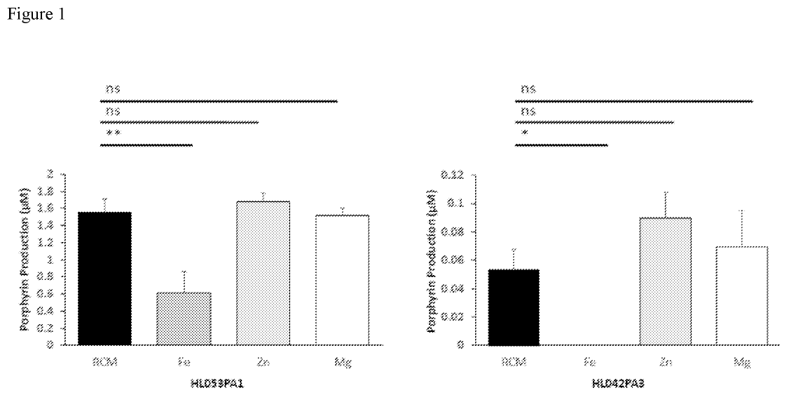

FIG. 2 shows that P. acnes strains respond to iron in media.

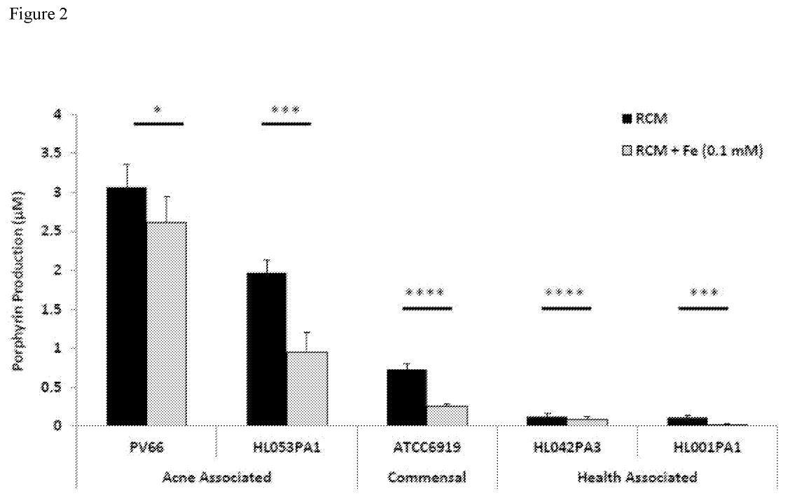

FIG. 3 shows that porphyrin production increases in response to reduced iron level by chelators in acne associated P. acnes strains.

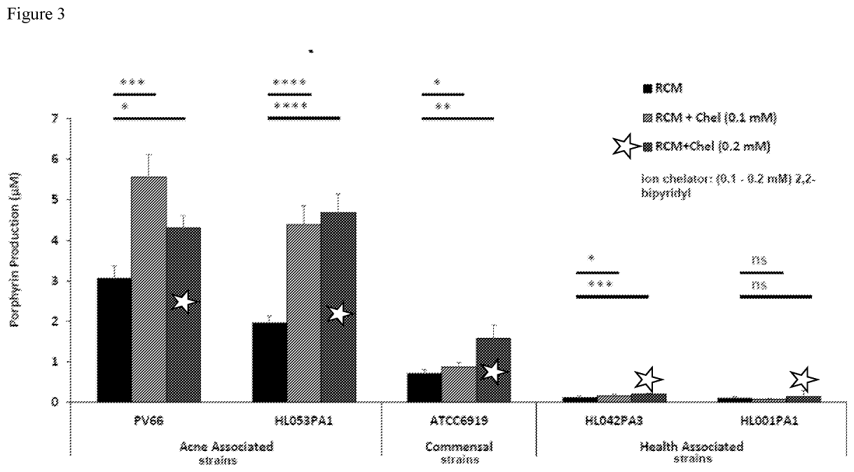

FIG. 4 shows that iron reduces porphyrin production in acne associated P. acnes strains. Iron added back to iron depleted (chelated) media diminishes chelator effect on porphyrin production in P. acnes strains.

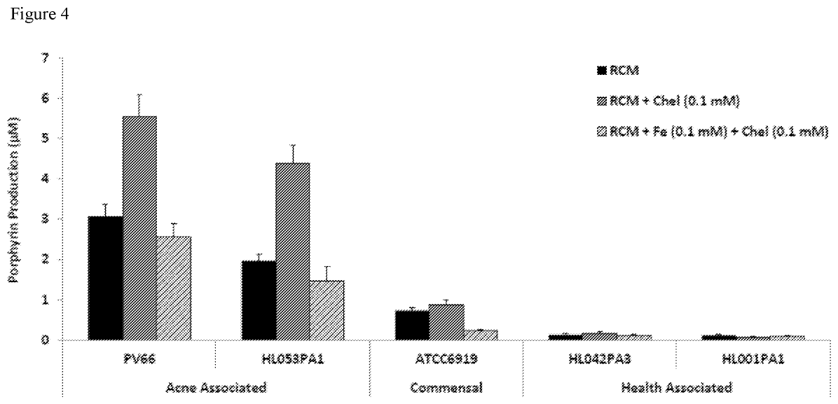

FIG. 5 shows that depletion of cobalt significantly promoted the porphyrin production in P. acnes.

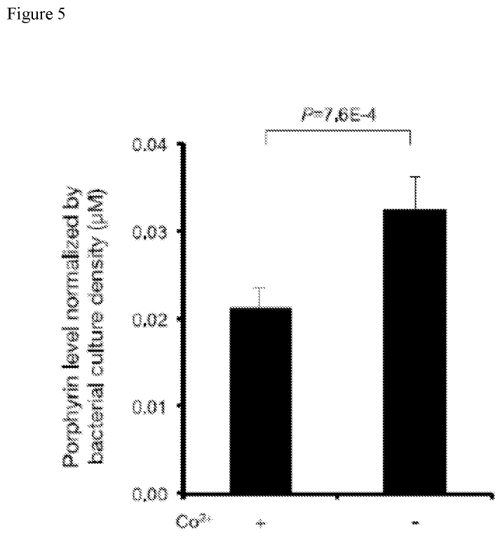

FIG. 6 shows iron depletion increases porphyrin production in acne associated P. acnes strains. This effect remains in vitamin B12 supplemented conditions.

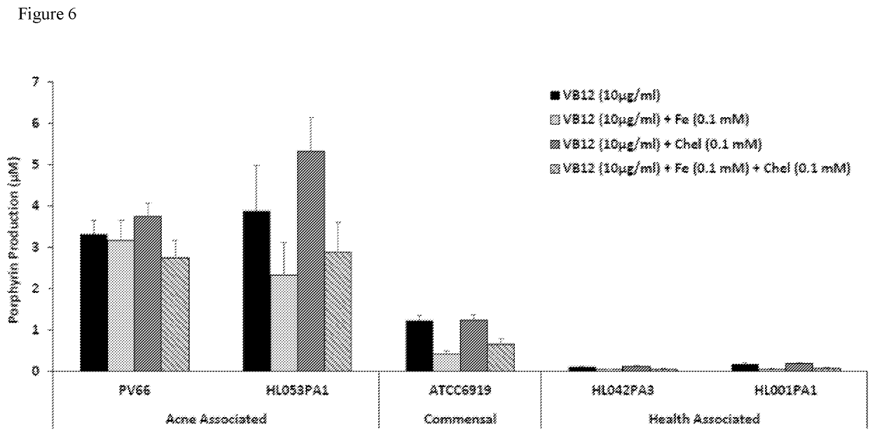

### DETAILED DESCRIPTION

In some aspects, provided herein are method and compositions (e.g., pharmaceutical or cosmetic compositions) useful in treating or preventing a skin disease or skin aging in a subject in need thereof by administering a composition comprising iron and/or cobalt to the subject. In some embodiments, the subject has a skin disease (e.g., inflammatory skin disease, such as acne, rosacea, or Porphyria Cutanea Tarda (PCT)).

In some aspects, provided herein are methods and compositions related to treating or preventing a skin disease (e.g., inflammatory skin disease, such as acne, rosacea, or Porphyria Cutanea Tarda (PCT)), preventing and/or slowing skin aging (e.g., preventing the formation of wrinkles), and maintaining healthy skin by administering to the subject a composition disclosed herein. The method of any one of the preceding claims, wherein the skin disease is an inflammatory skin disease. The skin disease may be acne, rosacea, or PCT.

In some aspects, provided herein are methods of treating or preventing a skin condition in a subject and/or reducing the amount of porphyrins on the skin of a subject by administering (e.g., a subject with symptoms of skin aging, or a subject with a skin condition, such as a skin disease associated with inflammation, acne, rosacea, or PCT) a composition disclosed herein to the subject. The porphyrins may be produced by P. acnes (e.g., acne-associated of P. acnes strains).

In some aspects, provided herein are methods of promoting vitamin B12 biosynthesis in acne-associated strains of P. acnes on the skin of a subject by administering a composition comprising cobalt and/or iron. In some embodiments, the subject has symptoms of skin aging. In some embodiments, the subject has a skin condition, such as a skin disease associated with inflammation, acne, rosacea, or PCT.

### Definitions

As used herein the specification, “a” or “an” may mean one or more. As used herein in the claim(s), when used in conjunction with the word “comprising”, the words “a” or “an” may mean one or more than one. As used herein “another” may mean at least a second or more.

The term “preventing” is art-recognized, and when used in relation to a condition, such as a local recurrence, is well understood in the art, and includes administration of a composition which reduces the frequency of, or delays the onset of, symptoms of a medical condition in a subject relative to a subject which does not receive the composition. Thus, prevention of acne includes, for example, reducing the number of detectable acne lesions in a population of patients receiving a prophylactic treatment relative to an untreated control population, and/or delaying the appearance of detectable lesions in a treated population versus an untreated control population, e.g., by a statistically and/or clinically significant amount.

The term “prophylactic” or “therapeutic” treatment is art-recognized and includes administration to the host of one or more of the subject compositions. If it is administered prior to clinical manifestation of the unwanted condition (e.g., disease or other unwanted state of the host animal) then the treatment is prophylactic (i.e., it protects the host against developing the unwanted condition), whereas if it is administered after manifestation of the unwanted condition, the treatment is therapeutic (i.e., it is intended to diminish, ameliorate, or stabilize the existing unwanted condition or side effects thereof).

The term “ribotype” refers to strains of P. acnes. The ribotyped strains were characterized as in Fitz-Gibbon et al. (J. Investigative Dermatology 133:2152-60 (2013)).

The term “subject” refers to a mammal, including, but not limited to, a human or non-human mammal, such as a bovine, equine, canine, ovine, or feline.

A “therapeutically effective amount” of a compound with respect to the subject method of treatment refers to an amount of the compound(s) in a preparation which, when administered as part of a desired dosage regimen (to a mammal, preferably a human) alleviates a symptom, ameliorates a condition, or slows the onset of disease conditions according to clinically acceptable standards for the disorder or condition to be treated or the cosmetic purpose, e.g., at a reasonable benefit/risk ratio applicable to any medical treatment.

As used herein, the term “treating” or “treatment” includes reversing, reducing, or arresting the symptoms, clinical signs, and underlying pathology of a condition in a manner to improve or stabilize a subject's condition.

### Therapeutic Methods

Provided herein are methods of treating or preventing a skin condition, preventing and/or slowing skin aging, and/or maintaining healthy skin. In some aspects, the methods relate to treating or preventing a skin disease in a subject, comprising administering a composition disclosed herein to the subject.

Compositions described herein comprise iron and/or cobalt. The compositions disclosed herein may further comprise one or more strains of P. acnes. The P. acnes strain may be a strain associated with healthy skin. The P. acnes strain may be RT1, RT2, RT3, or RT6 strain of P. acnes. The compositions may comprise at least one phage against a strain of P. acnes. The phage may target a strain of P. acnes that is associated with acne or an inflammatory skin disease, such as RT4, RT5, RT7, RT8, RT9 or RT10. The composition may comprise P. granulosum, P. avidum, P. humerusii. Compositions disclosed herein may comprise an antibiotic (e.g., an antibiotic that does not target P. granulosum, P. avidum, P. humerusii, or an RT1, RT2, RT3, or RT6 strain of P. acnes). Compositions may contain two or more, three or more, four or more, five or more, six or more, seven or more, eight or more, nine or more, or ten or more strains of P. acnes (e.g., an RT1, RT2, RT3, or RT6 strain of P. acnes), P. granulosum, P. avidum, and/or P. humerusii.

In some aspects, provided herein are methods related to reducing the amount of porphyrins on the skin of a subject, comprising administering a composition disclosed herein. In some embodiments, the subject has a skin disease or symptoms associated with skin aging (e.g., formation of wrinkles). In some embodiments, the skin disease is an inflammatory skin disease, such as acne, rosacea, or Porphyria Cutanea Tarda (PCT). In some aspects, provided herein are methods of slowing or preventing skin aging in a subject by administering a composition disclosed herein to the subject.

The strain of P. granulosum may be HL078PG1 or HL082PG1. The strain of P. avidum may be HL063PV1, HL083PV1, or HL307PV1. The strain of P. humerusii may be HL044PA1, HL037PA2, or HL037PA3.

In some embodiments, the composition further comprises a phage against a strain of P. acnes (e.g., a RT4, RT5, RT7, RT8, RT9 or RT10 strain of P. acnes). In some embodiments, the composition comprises two or more (e.g., three or more, four or more, five or more, six or more, seven or more, eight or more, nine or more, or ten or more) phages against a strain of P. acnes. The type of phage that may be administered in a composition disclosed herein depends on the type of acne or skin disease, the medical history of an individual, or the symptoms of a subject with a skin disease. Non-limiting examples of phages include PHL111M01, PHL082M00, PHL060L00, PHL067M10, PHL071N05, PHL112N00, PHL037M02, PHL085N00, PHL115M02, PHL085M01, PHL114L00, PHL010M04, PHL066M04, PHL071N05, PHL113M01, PHL112N00, and PHL037M02. Information about P. acnes phages can be found in U.S. Patent Publication US20150086581A1, hereby incorporated in its entirety. In some embodiments, the composition may comprise an antibiotic (e.g., an antibiotic that does not target P. granulosum, P. avidum, P. humerusii, or an RT1, RT2, RT3, or RT6 strain of P. acnes).

The compositions disclosed herein may be administered to a subject by any means known in the art, for example, the composition may be formulated for topical delivery. The formulation may be a liquid, gel, or cream. In some embodiments, the composition is formulated for oral delivery. The composition may be in the form of a pill, tablet, or capsule. In some embodiments, the subject may be a mammal (e.g., a human). In some embodiments, the composition is self-administered.

In general, the above methods directly act to reduce the amount of pathogenic bacteria in a subject. In general, the above methods directly act to reduce the amount of porphyrins produced by disease-associated bacteria in a subject. In some embodiments, this includes any such therapy that achieves the same goal of reducing the number of pathogenic organisms, when used in combination with the composition described herein, would lead to replacement of the pathogenic microflora involved in the diseased state with natural microflora enriched in skin not afflicted with a skin disease, or less pathogenic species occupying the same ecological niche as the type causing a disease state. For example, a subject may undergo treatment with antibiotics or a composition comprising antibiotics to target and decrease the prevalence of pathogenic or acne-associated organisms, and subsequently be treated with a composition described herein.

Suitable antimicrobial compounds include capreomycins, including capreomycin IA, capreomycin IB, capreomycin HA and capreomycin IIB; carbomycins, including carbomycin A; carumonam; cefaclor, cefadroxil, cefamandole, cefatrizine, cefazedone, cefazolin, cefbuperazone, cefcapene pivoxil, cefclidin, cefdinir, cefditoren, cefime, ceftamet, cefmenoxime, cefinetzole, cefminox, cefodizime, cefonicid, cefoperazone, ceforanide, cefotaxime, cefotetan, cefotiam, cefoxitin, cefpimizole, cefpiramide, cefpirome, cefprozil, cefroxadine, cefsulodin, ceftazidime, cefteram, ceftezole, ceftibuten, ceftiofur, ceftizoxime, ceftriaxone, cefuroxime, cefuzonam, cephalexin, cephalogycin, cephaloridine, cephalosporin C, cephalothin, cephapirin, cephamycins, such as cephamycin C, cephradine, chlortetracycline; chlarithromycin, clindamycin, clometocillin, clomocycline, cloxacillin, cyclacillin, danofloxacin, demeclocyclin, destomycin A, dicloxacillin, dirithromycin, doxycyclin, epicillin, erythromycin A, ethanbutol, fenbenicillin, flomoxef, florfenicol, floxacillin, flumequine, fortimicin A, fortimicin B, forfomycin, foraltadone, fusidic acid, gentamycin, glyconiazide, guamecycline, hetacillin, idarubicin, imipenem, isepamicin, josamycin, kanamycin, leumycins such as leumycin A1, lincomycin, lomefloxacin, loracarbef, lymecycline, meropenam, metampicillin, methacycline, methicillin, mezlocillin, micronomicin, midecamycins such as midecamycin A1, mikamycin, minocycline, mitomycins such as mitomycin C, moxalactam, mupirocin, nafcillin, netilicin, norcardians such as norcardian A, oleandomycin, oxytetracycline, panipenam, pazufloxacin, penamecillin, penicillins such as penicillin G, penicillin N and penicillin O, penillic acid, pentylpenicillin, peplomycin, phenethicillin, pipacyclin, piperacilin, pirlimycin, pivampicillin, pivcefalexin, porfiromycin, propiallin, quinacillin, ribostamycin, rifabutin, rifamide, rifampin, rifamycin SV, rifapentine, rifaximin, ritipenem, rekitamycin, rolitetracycline, rosaramicin, roxithromycin, sancycline, sisomicin, sparfloxacin, spectinomycin, streptozocin, sulbenicillin, sultamicillin, talampicillin, teicoplanin, temocillin, tetracyclin, thostrepton, tiamulin, ticarcillin, tigemonam, tilmicosin, tobramycin, tropospectromycin, trovafloxacin, tylosin, and vancomycin, and analogs, derivatives, pharmaceutically acceptable salts, esters, prodrugs, and protected forms thereof.

Suitable anti-fungal compounds include ketoconazole, miconazole, fluconazole, clotrimazole, undecylenic acid, sertaconazole, terbinafine, butenafine, clioquinol, haloprogin, nystatin, naftifine, tolnaftate, ciclopirox, amphotericin B, or tea tree oil and analogs, derivatives, pharmaceutically acceptable salts, esters, prodrugs, and protected forms thereof.

Suitable antiviral agents include acyclovir, azidouridine, anismoycin, amantadine, bromovinyldeoxusidine, chlorovinyldeoxusidine, cytarabine, delavirdine, didanosine, deoxynojirimycin, dideoxycytidine, dideoxyinosine, dideoxynucleoside, desciclovir, deoxyacyclovir, efavirenz, enviroxime, fiacitabine, foscamet, fialuridine, fluorothymidine, floxuridine, ganciclovir, hypericin, idoxuridine, interferon, interleukin, isethionate, nevirapine, pentamidine, ribavirin, rimantadine, stavudine, sargramostin, suramin, trichosanthin, tribromothymidine, trichlorothymidine, trifluorothymidine, trisodium phosphomonoformate, vidarabine, zidoviridine, zalcitabine and 3-azido-3-deoxythymidine and analogs, derivatives, pharmaceutically acceptable salts, esters, prodrugs, and protected forms thereof.

Other suitable antiviral agents include 2′,3′-dideoxyadenosine (ddA), 2′,3′-dideoxyguanosine (ddG), 2′,3′-dideoxycytidine (ddC), 2′,3′-dideoxythymidine (ddT), 2′3′-dideoxy-dideoxythymidine (d4T), 2′-deoxy-3′-thia-cytosine (3TC or lamivudime), 2′,3′-dideoxy-2′-fluoroadenosine, 2′,3′-dideoxy-2′-fluoroinosine, 2′,3′-dideoxy-2′-fluorothymidine, 2′,3′-dideoxy-2′-fluorocytosine, 2′3′-dideoxy-2′,3′-didehydro-2′-fluorothymidine (Fd4T), 2′3′-dideoxy-2′-beta-fluoroadenosine (F-ddA), 2′3′-dideoxy-2′-beta-fluoro-inosine (F-ddI), and 2′,3′-dideoxy-2′-beta-flurocytosine (F-ddC). In some embodiments, the antiviral agent is selected from trisodium phosphomonoformate, ganciclovir, trifluorothymidine, acyclovir, 3′-azido-3′-thymidine (AZT), dideoxyinosine (ddI), and idoxuridine and analogs, derivatives, pharmaceutically acceptable salts, esters, prodrugs, and protected forms thereof.

### Pharmaceutical Compositions

In some aspects, the invention relates to a composition (e.g., a pharmaceutical or cosmetic composition comprising iron and/or cobalt) disclosed herein. The pharmaceutical composition may be formulated for topical administration. The pharmaceutical composition may be a probiotic. The pharmaceutical compositions disclosed herein may be delivered by any suitable route of administration, including orally, buccally, sublingually, parenterally, and topically, as by powders, ointments, drops, liquids, gels, or creams. In certain embodiments, the pharmaceutical compositions are delivered generally (e.g., via oral or parenteral administration). In certain other embodiments, the pharmaceutical compositions are delivered locally through injection.

Actual dosage levels of the active ingredients in the pharmaceutical compositions may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular agent employed, the route of administration, the time of administration, the rate of excretion or metabolism of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could prescribe and/or administer doses of the compounds employed in the pharmaceutical composition at levels lower than that required in order to achieve the desired therapeutic effect and gradually increase the dosage until the desired effect is achieved.

Exemplary identities of various constituents of the topical formulations of some embodiments of the present invention are described below.

### Vehicles

Suitable topical vehicles and vehicle components for use with the formulations of the invention are well known in the cosmetic and pharmaceutical arts, and include such vehicles (or vehicle components) as water; organic solvents such as alcohols (particularly lower alcohols readily capable of evaporating from the skin such as ethanol), glycols (such as propylene glycol, butylene glycol, and glycerol (glycerin)), aliphatic alcohols (such as lanolin); mixtures of water and organic solvents (such as water and alcohol), and mixtures of organic solvents such as alcohol and glycerol (optionally also with water); lipid-based materials such as fatty acids, acylglycerols (including oils, such as mineral oil, and fats of natural or synthetic origin), phosphoglycerides, sphingolipids and waxes; protein-based materials such as collagen and gelatin; silicone-based materials (both non-volatile and volatile) such as cyclomethicone, dimethiconol, dimethicone, and dimethicone copolyol; hydrocarbon-based materials such as petrolatum and squalane; and other vehicles and vehicle components that are suitable for administration to the skin, as well as mixtures of topical vehicle components as identified above or otherwise known to the art.

In one embodiment, the compositions of the present invention are oil-in-water emulsions. Liquids suitable for use in formulating compositions of the present invention include water, and water-miscible solvents such as glycols (e.g., ethylene glycol, butylene glycol, isoprene glycol, propylene glycol), glycerol, liquid polyols, dimethyl sulfoxide, and isopropyl alcohol. One or more aqueous vehicles may be present.

In some embodiments, formulations do not have methanol, ethanol, propanols, or butanol.

### Surfactants and Emulsifiers

Many topical formulations contain chemical emulsions which use surface active ingredients (emulsifiers and surfactants) to disperse dissimilar chemicals in a particular solvent system. For example, most lipid-like (oily or fatty) or lipophilic ingredients do not uniformly disperse in aqueous solvents unless they are first combined with emulsifiers, which form microscopic aqueous soluble structures (droplets) that contain a lipophilic interior and a hydrophilic exterior, resulting in an oil-in-water emulsion. In order to be soluble in aqueous media, a molecule must be polar or charged so as to favorably interact with water molecules, which are also polar. Similarly, to dissolve an aqueous-soluble polar or charged ingredient in a largely lipid or oil-based solvent, an emulsifier is typically used which forms stable structures that contain the hydrophilic components in the interior of the structure while the exterior is lipophilic so that it can dissolve in the lipophilic solvent to form a water-in-oil emulsion. It is well known that such emulsions can be destabilized by the addition of salts or other charged ingredients which can interact with the polar or charged portions of the emulsifier within an emulsion droplet. Emulsion destabilization results in the aqueous and lipophilic ingredients separating into two layers, potentially destroying the commercial value of a topical product.

Surfactants suitable for use in the present invention may be ionic or non-ionic. These include, but are not limited to: cetyl alcohol, polysorbates (Polysorbate 20, Polysorbate 40, Polysorbate 60, Polysorbate 80), steareth-10 (Brij 76), sodium dodecyl sulfate (sodium lauryl sulfate), lauryl dimethyl amine oxide, cetyltrimethylammonium bromide (CTAB), polyethoxylated alcohols, polyoxyethylene sorbitan, octoxynol, N,N-dimethyldodecylamine-N-oxide, hexadecyltrimethylammonium bromide (HTAB), polyoxyl 10 lauryl ether, bile salts (such as sodium deoxycholate or sodium cholate), polyoxyl castor oil, nonylphenol ethoxylate, cyclodextrins, lecithin, dimethicone copolyol, lauramide DEA, cocamide DEA, cocamide MEA, oleyl betaine, cocamidopropyl betaine, cocamidopropyl phosphatidyl PG-dimonium chloride, dicetyl phosphate (dihexadecyl phosphate), ceteareth-10 phosphate, methylbenzethonium chloride, sodium methyl cocoyl taurate, decyl glucoside, sodium cocoyl glutamate, dicetyl phosphate, ceteth-10 phosphate (ceteth-10 is the polyethylene glycol ether of cetyl alcohol where n has an average value of 10; ceteth-10 phosphate is a mixture of phosphoric acid esters of ceteth-10), ceteth-20, Brij S10 (polyethylene glycol octadecyl ether, average Mₙ˜711), and Poloxamers (including, but not limited to, Poloxamer 188 (HO(C₂H₄O)ₐ(CH(CH₃)CH₂O)_{b}(C₂H₄O)ₐH, average molecular weight 8400) and Poloxamer 407 (HO(C₂H₄O)ₐ(CH(CH₃)CH₂O)_{b}(C₂H₄O)ₐH, wherein a is about 101 and b is about 56)). Appropriate combinations or mixtures of such surfactants may also be used according to the present invention. Many of these surfactants may also serve as emulsifiers in formulations of the present invention.

Other suitable emulsifiers for use in the formulations of the present invention include, but are not limited to, behentrimonium methosulfate-cetearyl alcohol, non-ionic emulsifiers like emulsifying wax, polyoxyethylene oleyl ether, PEG-40 stearate, cetostearyl alcohol (cetearyl alcohol), ceteareth-12, ceteareth-20, ceteareth-30, ceteareth alcohol, Ceteth-20 (Ceteth-20 is the polyethylene glycol ether of cetyl alcohol where n has an average value of 20), oleic acid, oleyl alcohol, glyceryl stearate, PEG-75 stearate, PEG-100 stearate, and PEG-100 stearate, ceramide 2, ceramide 3, stearic acid, cholesterol, steareth-2, and steareth-20, or combinations/mixtures thereof, as well as cationic emulsifiers like stearamidopropyl dimethylamine and behentrimonium methosulfate, or combinations/mixtures thereof.

### Moisturizers, Emollients, and Humectants

One of the most important aspects of topical products in general, and cosmetic products in particular, is the consumer's perception of the aesthetic qualities of a product. For example, while white petrolatum is an excellent moisturizer and skin protectant, it is rarely used alone, especially on the face, because it is greasy, sticky, does not rub easily into the skin and may soil clothing. Consumers highly value products which are aesthetically elegant and have an acceptable tactile feel and performance on their skin. Suitable moisturizers for use in the formulations of the present invention include, but are not limited to, lactic acid and other hydroxy acids and their salts, glycerol, propylene glycol, butylene glycol, sodium PCA, sodium hyaluronate, Carbowax 200, Carbowax 400, and Carbowax 800. Suitable emollients or humectants for use in the formulations of the present invention include, but are not limited to, acemannan, derivatives of Aloe vera, panthenol, N-palmitoylethanolamine, N-acetylethanolamine, cetyl palmitate, glycerol (glycerin), PPG-15 stearyl ether, lanolin alcohol, lanolin, lanolin derivatives, cholesterol, petrolatum, isostearyl neopentanoate, octyl stearate, mineral oil, isocetyl stearate, myristyl myristate, octyl dodecanol, 2-ethylhexyl palmitate (octyl palmitate), dimethicone, phenyl trimethicone, cyclomethicone, C₁₂-C₁₅ alkyl benzoates, dimethiconol, propylene glycol, Theobroma grandiflorum seed butter, ceramides (e.g., ceramide 2 or ceramide 3), hydroxypropyl bispalmitamide MEA, hydroxypropyl bislauramide MEA, hydroxypropyl bisisostearamide MEA, 1,3-bis(N-2-(hydroxyethyl)stearoylamino)-2-hydroxy propane, bis-hydroxyethyl tocopherylsuccinoylamido hydroxypropane, urea, aloe, allantoin, glycyrrhetinic acid, safflower oil, oleyl alcohol, oleic acid, stearic acid, dicaprylate/dicaprate, diethyl sebacate, isostearyl alcohol, pentylene glycol, isononyl isononanoate, and 1,3-bis(N-2-(hydroxyethyl)palmitoylamino)-2-hydroxypropane. In addition, appropriate combinations and mixtures of any of these moisturizing agents and emollients may be used in accordance with the present invention.

### Preservatives and Antioxidants

The composition may further include components adapted to improve the stability or effectiveness of the applied formulation.

Suitable preservatives for use in the present invention include, but are not limited to: ureas, such as imidazolidinyl urea and diazolidinyl urea; phenoxyethanol; sodium methyl paraben, methylparaben, ethylparaben, and propylparaben; potassium sorbate; sodium benzoate; sorbic acid; benzoic acid; formaldehyde; citric acid; sodium citrate; chlorine dioxide; bakuchiol, N-acetyl-L-cysteine, honokiol, magnolol, derivatives of Magnolia officinalis bark, chrysin, quaternary ammonium compounds, such as benzalkonium chloride, benzethonium chloride, cetrimide, dequalinium chloride, and cetylpyridinium chloride; mercurial agents, such as phenylmercuric nitrate, phenylmercuric acetate, and thimerosal; piroctone olamine; Vitis vinifera seed oil; and alcoholic agents, for example, chlorobutanol, dichlorobenzyl alcohol, phenylethyl alcohol, and benzyl alcohol.

Suitable antioxidants include, but are not limited to, ascorbic acid and its esters, sodium bisulfite, butylated hydroxytoluene, D-ribose, N-acetyl-L-cysteine, apocynin, bixin, derivatives of Bixa orellana seeds, nicotinamide, acetyl zingerone, bakuchiol, tiliroside, extracts of Fragraria ananassa seed, fucoxanthin, creatine and its salts and esters, quercetin, luteolin, honokiol, magnolol, derivatives of Magnolia officinalis bark, butylated hydroxyanisole, tocopherols, tocopheryl acetate, sodium ascorbate/ascorbic acid, ascorbyl palmitate, propyl gallate, and chelating agents like EDTA (e.g., disodium EDTA), citric acid, and sodium citrate.

In some embodiments, the antioxidant or preservative comprises (3-(4-chlorophenoxy)-2-hydroxypropyl)carbamate.

In some embodiments, antioxidants or preservatives of the present invention may also function as a moisturizer or emollient, for example.

In addition, combinations or mixtures of these preservatives or anti-oxidants may also be used in the formulations of the present invention.

### Combination Agents

The composition can also contain any other agent that has a desired effect when applied topically to a subject. Suitable classes of active agents include, but are not limited to antibiotic agents (i.e., an antibiotic that does not target P. granulosum, P. avidum, P. humerusii, or an RT1, RT2, RT3, or RT6 strain of P. acnes), antimicrobial agents, anti-acne agents, antibacterial agents, antifungal agents, antiviral agents, steroidal anti-inflammatory agents, non-steroidal anti-inflammatory agents, anesthetic agents, antipruriginous agents, antiprotozoal agents, anti-oxidants, antihistamines, vitamins, and hormones. Mixtures of any of these active agents may also be employed. Additionally, dermatologically-acceptable salts and esters of any of these agents may be employed.

### Viscosity Modifiers

Suitable viscosity adjusting agents (i.e., thickening and thinning agents or viscosity modifying agents) for use in the formulations of the present invention include, but are not limited to, protective colloids or non-ionic gums such as hydroxyethylcellulose, xanthan gum, and sclerotium gum, as well as magnesium aluminum silicate, silica, microcrystalline wax, beeswax, paraffin, and cetyl palmitate. In addition, appropriate combinations or mixtures of these viscosity adjusters may be utilized according to the present invention.

### Additional Constituents

Additional constituents suitable for incorporation into the emulsions of the present invention include, but are not limited to: skin protectants, adsorbents, demulcents, emollients, moisturizers, sustained release materials, solubilizing agents, skin-penetration agents, skin soothing agents, deodorant agents, antiperspirants, sun screening agents, sunless tanning agents, vitamins, hair conditioning agents, anti-irritants, anti-aging agents, abrasives, absorbents, anti-caking agents, anti-static agents, astringents (e.g., witch hazel, alcohol, and herbal extracts such as chamomile extract), binders/excipients, buffering agents, chelating agents, film forming agents, conditioning agents, opacifying agents, lipids, immunomodulators, and pH adjusters (e.g., citric acid, sodium hydroxide, and sodium phosphate). For example, lipids normally found in healthy skin (or their functional equivalents) may be incorporated into the emulsions of the present invention. In certain embodiments, the lipid is selected from the group consisting of ceramides, cholesterol, and free fatty acids. Examples of lipids include, but are not limited to, ceramide 1, ceramide 2, ceramide 3, ceramide 4, ceramide 5, ceramide 6, hydroxypropyl bispalmitamide MEA, and hydroxypropyl bislauramide MEA, and combinations thereof.

Examples of peptides that interact with protein structures of the dermal-epidermal junction include palmitoyl dipeptide-5 diaminobutyloyl hydroxythreonine and palmitoyl dipeptide-6 diaminohydroxybutyrate.

Examples of skin soothing agents include, but are not limited to algae extract, mugwort extract, stearyl glycyrrhetinate, bisabolol, allantoin, aloe, avocado oil, green tea extract, hops extract, chamomile extract, colloidal oatmeal, calamine, cucumber extract, and combinations thereof.

In certain embodiments, the compositions comprise bergamot or bergamot oil. Bergamot oil is a natural skin toner and detoxifier. In certain embodiments, it may prevent premature aging of skin and may have excellent effects on oily skin conditions and acne.

In some embodiments, the composition comprises a vitamin. Examples of vitamins include, but are not limited to, vitamins A, D, E, K, and combinations thereof. Vitamin analogues are also contemplated; for example, the vitamin D analogues calcipotriene or calcipotriol. In some embodiments, the vitamin may be present as tetrahexyldecyl ascorbate. This compound exhibits anti-oxidant activity, inhibiting lipid peroxidation. In certain embodiments, use can mitigate the damaging effects of UV exposure. Studies have shown it to stimulate collagen production as well as clarifying and brightening the skin by inhibiting melanogenesis (the production of pigment) thereby promoting a more even skin tone.

In some embodiments, the composition comprises a sunscreen. Examples of sunscreens include, but are not limited to, p-aminobenzoic acid, avobenzone, cinoxate, dioxybenzone, homosalate, menthyl anthranilate, octocrylene, octyl methoxycinnamate, octyl salicylate, oxybenzone, padimate O, phenylbenzimidazole sulfonic acid, sulisobenzone, titanium dioxide, trolamine salicylate, zinc oxide, 4-methylbenzylidene camphor, methylene bis-benzotriazolyl tetramethylbutylphenol, bis-ethylhexyloxyphenol methoxyphenyl triazine, terephthalylidene dicamphor sulfonic acid, drometrizole trisiloxane, disodium phenyl dibenzimidazole tetrasulfonate, diethylamino hydroxybenzoyl hexyl benzoate, octyl triazone, diethylhexyl butamido triazone, polysilicone-15, and combinations thereof.

Suitable fragrances and colors may be used in the formulations of the present invention. Examples of fragrances and colors suitable for use in topical products are known in the art.

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

### EQUIVALENTS

Those skilled in the art will recognize, or be able to ascertain using no more than routine experimentation, many equivalents to the specific embodiments are described herein. Such equivalents are intended to be encompassed by the following claims.

What is claimed is:

1. A method of treating or preventing a skin disease or skin aging in a subject in need thereof, comprising administering a composition comprising iron to the subject.

2. The method of claim of claim 1, wherein the composition further comprises cobalt.

3. A method of treating or preventing a skin disease or skin aging in a subject in need thereof, comprising administering a composition comprising cobalt to the subject.

4. The method of claim of claim 3, wherein the composition further comprises iron.

5. The method of any one of the preceding claims, wherein the skin disease is an inflammatory skin disease.

6. The method of any one of the preceding claims, wherein the skin disease is acne.

7. The method of any one of claims 1 to 5, wherein the skin disease is rosacea.

8. The method of any one of claims 1 to 5, wherein the skin disease is Porphyria Cutanea Tarda (PCT).

9. A method of reducing the amount of porphyrins on the skin of a subject, comprising administering a composition comprising iron to the subject.

10. A method of reducing the amount of porphyrins on the skin of a subject, comprising administering a composition comprising cobalt to the subject.

11. The method of claim 9 or 10, wherein the porphyrins are produced by P. acnes.

12. The method of claim 11, wherein the porphyrins are produced by acne-associated strains of P. acnes.

13. The method of any one of claims 9 to 12, wherein the subject has a skin disease or symptoms associated with skin aging.

14. The method of any one of claims 9 to 13, wherein the subject has a skin disease associated with inflammation.

15. The method of any one of claims 9 to 14, wherein the subject has acne.

16. The method of any one of claims 9 to 14, wherein the subject has rosacea.

17. The method of any one of claims 9 to 14, wherein the subject has Porphyria Cutanea Tarda (PCT).

18. The method of any one of the preceding claims, wherein the composition further comprises one or more strains of P. acnes.

19. The method of claim 18, wherein the P. acnes strain is a RT1, RT2, RT3, or RT6 strain of P. acnes.

20. The method of any one of the preceding claims, wherein the composition also comprises at least one phage against a strain of P. acnes.

21. The method of claim 20, wherein the phage targets a strain of P. acnes selected from RT4, RT5, RT7, RT8, RT9 or RT10.

22. The method of any one of the preceding claims, wherein the composition further comprises P. granulosum.

23. The method of any one of the preceding claims, wherein the composition further comprises P. avidum.

24. The method of any one of the preceding claims, wherein the composition further comprises P. humerusii.

25. The method of any one of the preceding claims, wherein the composition further comprises an antibiotic.

26. The method of any one of the preceding claims, wherein the composition is formulated for topical delivery.

27. The method of any one of the preceding claims, wherein the composition is formulated for oral delivery.

28. The method of any one of the preceding claims, wherein the subject is a human.

29. A pharmaceutical or cosmetic composition comprising iron.

30. A pharmaceutical or cosmetic composition comprising cobalt.

31. The composition of claim 29 or 30, wherein the composition further comprises at least one strain of P. acnes.

32. The composition of claim 31, wherein the at least one strain of P. acnes is RT1 or RT6.

33. The composition of claim 31, wherein the at least one strain of P. acnes is RT2 or RT3.

34. The composition of any one of claims 29 to 33, wherein the composition comprises a phage against RT4, RT5, RT7, RT8, RT9 or a RT10 strain of P. acnes.

35. The composition of any one of claims 29 to 34, wherein the composition further comprises P. granulosum.

36. The composition of any one of claims 29 to 35, wherein the composition further comprises P. avidum.

37. The composition of any one of claims 29 to 36, wherein the composition further comprises P. humerusii.

38. A method for treating or preventing a skin disease, comprising administering to the subject the composition of any one of claims 29 to 37.

39. A method for slowing or preventing skin aging, comprising administering to the subject the composition of any one of claims 29 to 37.

40. The method of claim 38 or 39, wherein the composition further comprises an antibiotic.

41. The method of any one of claims 38 to 40, wherein the composition is administered topically.

42. The method of any one of claims 38 to 40, wherein the composition is administered orally.
